(19) United States
(12) Patent Application Publication
(10) Pub. No.: US 2004/0023953 A1
(43) Pub. Date: Feb. 5, 2004

(54) Tricylic mercaptomethyl-substituted 2,3-dihydro-quinazolin-5-ones and 2,3-dihydro-benzo-[1,2,4]-thiadiazin-5,5-dioxides as matrix metalloproteinase (MMP) inhibitors

(76) Inventors: Uwe Kausmeier, Guersids (DE); Jochen Heinicke, Leipzig (DE); Christoph Arkona, Leipzig (DE)

Correspondence Address: BURNS DOANE SWECKER & MATHIS L L P, POST OFFICE BOX 1404, ALEXANDRIA, VA, 22313-1404, US

(21) Appl. No.: 10/381,024

(22) PCT Filed: Jul. 5, 2001
(86) PCT No.: PCT/EP01/07710

(30) Foreign Application Priority Data
Sep. 21, 2000 (DE) 100 46 728.8

(51) Int. Cl. A61K 31/542; A61K 31/549; A61K 31/519

(52) U.S. Cl. 514/222.8; 514/267; 544/9; 544/250

(57) ABSTRACT

The invention relates to mercaptomethyl-substituted thiazolo[2,3-b]quinazolines, oxazolo[2,3-b]quinazolines and imidazolo[2,1-b]quinazolines.

It is the object of the invention to discover new matrix metalloproteinase inhibitors (MMP) and give a simple synthesis method.

The object is achieved by the production of the indicated compounds. By means of them it is possible to achieve progress using them as active substances in pharmaceutical preparations for treating diseases such as inflammatory responses of non-specific causes, sunburn and allergies.

The present invention relates to representatives of the substance classes of the thiazolo [2,3-b]quinazolinones, oxazolo [2,3-b]quinazolinones, imidazo [2,1-b]quinazolinones as well as the benzothiadiazine-3,5-dioxides of general formulae Ia and Ib,

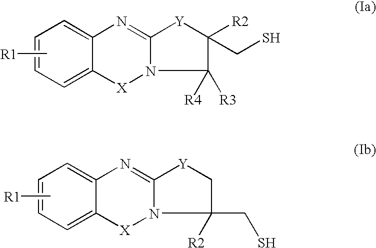

wherein

R1=hydrogen, methyl, methoxy, dimethoxy, Hal (Hal=fluorine, chlorine, bromine), carboxy

R2, R3, R4=hydrogen, methyl,

X=carbonyl, sulfonyl

Y=S, O, NH

and their tautomers and salts as well as to their use as matrix metalloproteinase (MMP) inhibitors.

It is the objective of this invention to develop medicaments for treating numerous diseases, such as cancer, rheumatism, non-specific inflammatory responses occurring inter alia in the case of sunburn and allergic reactions.

Quinazolines, quinazolinones and quinazolinediones are the subject of intensive pharmaceutical research. Their suitability as active substances and synthetic building blocks is undisputed.

As compared thereto, not much is known about the syntheses and effects of mercaptoalkyl-substituted quinazolinones and their bioisosteric analogues belonging to the group consisting of benzothiadiazine-1,1-dioxides. The effectiveness of sulfur-substituted quinazolinediones, especially of 3-(mercaptoalkyl)quinazoline-2,4-(1H,3H)-diones as pharmaceuticals was discovered by Leistner et al. (DD 298 784). It was found that an immunostimulatory and antiviral effectiveness of representatives of this substance group is given.

Compounds having a tricyclic basic system corresponding to formulae Ia and Ib and a different substitution pattern were described occasionally and showed special pharmacological effects (Cherr., J. -W. et al.: Studies on Quinazolines and 1,2,4-benzothiadiazines 1,1-dioxides. 8. Synthesis and pharmacological evaluation of tricyclic fused quinazolines and 1,2,4-benzothiadiazine 1,1-dioxides as potential α1-adrenoceptor antagonists, J. Med. Chem. 41, (1998), 3123-3141; Liu, K. -C. and Hsu, L. -Y.: Synthese and antihypertensive Aktivität einiger Chinazolinon-Darivate [synthesis and antihypertensive activity of some quinazoline derivatives], Arch. Pharm. 318 (1985) 502-505). Mercaptoalkyl-substituted tricyclene corresponding to general formulae Ia and Ib, however, have not yet been described in the technical literature.

Intensive research is being made in the field concerning the development of efficient, low-molecular and non-proteinogenous MMP inhibitors world-wide. It is known that from the physiological view-point the enzymatic activity of MMPs is subject to a strict, coordinated regulation between activation and inhibition. For this purpose, the organism has special proteins, what is called the tissue inhibitors of matrix metalloproteinases (TIMPs), which can inhibit rapidly and efficiently the activity of MMPs (Nagase, H. et al.: Engineering of selective TIMPS, 1-11 In: Inhibition of Matrix Metalloproteinase—Therapeutic Application (Eds. Greenwald, R. A., Zucker, S., Golub, L. M.) Ann NY Acad Sci 878 (1999). In particular in the case of the rheumatic diseases, an unblocked enzymatic activity of these enzymes results in the degradation of the cartilage substance and in chronic and painful changes of the joints, which is pathologically significant (Goldbach-Mansky, R. et al.: Active synovial matrix metalloproteinase-2 is associated with radiographic erosions in patients with early synovitis. Arthritis Res 2 (2000) 145-;53).

The invasion and spread of tumors represents another example of the pathological effect of MMPs. Released and activated MMPs force their way through the dense collagenous connective tissue and in particular also through the basal membrane of the vessels, thus making it possible for the cancer cells to leave the tumor aggregation, migrate into the vessel system and form metastases elsewhere. MMPs also play a decisive role for the blood vessel supply of the growing tumor by forcing the way for the newly formed blood vessels through the collagenous connective tissue, thus being responsible for this vascularization of the growing tumor (Shapiro, S. D.: Matrix metalloproteinase degradation of extracellular matrix: biological consequences. Current Opinion in Cell Biology 10 (1998) 602-608).

The U.V.-induced erythema is to be mentioned as another relevant medical result or an inadequately slowed-down effect of MMPs. It occurs inter alia as a result of intensive solar radiation. The high-energy U.V. rays of sunlight or of tanning devices activate inter alia the inactive procollagenases in the irradiated skin, which as a consequence cleave collagen of the connective tissue and the blood capillaries, thus being responsible for the symptoms of a sunburn.

With these illustrative pathological effects of the unrestrained enzymatic MPP action, the consequences thereof may be prevented or be reduced substantially by stable MMP inhibitors. It is fascinating to realize the idea of inhibiting these enzymes in well-calculated fashion by specific inhibitors to thus stop e.g. a progressive cartilage destruction occurring in connection with a disease of the rheumatic form or prevent the growth or spread of tumors.

Numerous methods of obtaining compounds having an MMP-inhibiting effect are already known. These first generation active substances usually have a proteinogenous structure and are structurally related to natural inhibitors which are special proteins. These proteinogenous or pseudo-proteinogenous substrate analogues have as a structural element a zinc-binding group chelating the zinc ion in the active MMP center.

As to a therapeutic application, all such proteinogenous and pseudo-proteinogenous active substances have a number of drawbacks, such as insufficient absorbability, usually short half-lives, only little stability as well as often undesired side-effects (Inhibition of Matrix Metalloproteinases-Therapeutic Application (Eds. Greenwald, R. A., Zucker, S., Golub, L. M., Ann NY Acad Sci 878 (1999)).

Further developments made in this field yielded e.g. phosphonamide inhibitors, piperazine inhibitors, sulfonamide inhibitors, carbamate inhibitors, diazepine inhibitors, tetracycline inhibitors and, last but not least, hydroxamate inhibitors (Skotnicki, J. S. at al.: Design and synthetic considerations of matrix metalloproteinase inhibitors, 61-72. In: Inhibition of Matrix Metalloproteinases-Therapeutic Application (Eds. Greenwald, R. A., Zucker, S., Golub, L. M.) Ann NY Acad Sci 878 (1999)). Although most of these developed inhibitors have impressive in vitro inhibitory effects and specificities, they showed a number of serious drawbacks in animal experiments and in humans when used in vivo. Here, cytotoxic reactions to a plurality of cells, a poor bioavailability and undesired side-effects, in particular a negative influence on the locomotor apparatus were to the fore.

Therefore, there is a demand for medicaments having a non-proteinogenous structure, which do not have the drawbacks of the active substances available thus far. In particular, there is a demand for new active substances which have an MMP-inhibitory effect, adequate stability and good absorbability, better pharmacokinetic properties and above all no undesired side-effects and cytotoxic reactions. It is thus the object of the present invention to discover new chemical substances of non-proteinogenous structure which display an MMP-inhibitory effect. It is a further object of this invention to provide methods of producing such compounds and corresponding medicaments which contain said compounds.

This object is achieved according to the claims.

The incentive compounds, produced for the first time, or general formulae Ia and Ib belonging to the class of tricyclic mercaptoalkyl-substituted 2,3-dihydroquinazoline-5-ones and their bioisosteric 2,3-dihydro-benzo-[1,2,4-]-thiadiazine-5,5-dioxides show surprising, marked and thus pharmacologically interesting MMP-inhibitory effects which cannot be derived from formerly known relationships between structure and effect.

Specificities of the compounds according to the invention are proved by testing these inhibitors corresponding to general formulae Ia and Ib using different human MMPs (MMP-2, recombinant catalytic domain of MMP-8, MMP-9, recombinant catalytic domain of MMP-14). In particular when used as medicaments, specific inhibitors would only influence the desired target enzymes but not disturb the balanced interplay of the other MMPs, thus helping to avoid possible undesired side-effects.

The inventive mercaptanes of general formulae Ia and Ib are obtained according to methods known in the literature by reacting the analogous iodides, bromides, chlorides or tosylates of general formula IIa or IIb

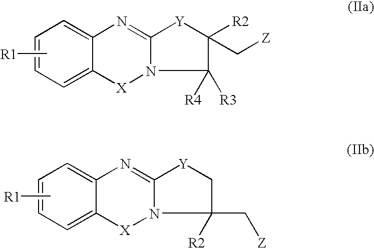

wherein:

R1=hydrogen, methyl, methoxy, dimethoxy, Hal (Hal=fluorine, chlorine, bromine), carboxy

R2, R3, R4=hydrogen, methyl

X=carbonyl, sulfonyl

Y=S, O, NH

Z=chlorine, bromine, iodine, tosyl

and the tautomers and salts thereof

with a sulfur-transferring agent, preferably thiourea, in an inert solvent and subsequent mild saponification of the isolated intermediate stages, preferably the isothiuronium salts.

The intermediate stages of general formulae IIa and IIb are known in the literature and/or obtainable according to synthetic principles known in the literature (Chem. J. -W. et al.: Studies on Quinazolines and 1,2,4-benzothiadiazine 1,1-dioxides, 8. Synthesis and pharmacological evaluation of tricyclic fused quinazolines and 1,2,4-benzothiadiazine 1,1-dioxides as potential α1-adrenoceptor Antagonists, J. Med. Chem. 41 (1998) 3128-3141; Shiau, C. -Y. et al., Studies on Quinazolines, 2. Synthesis of 2-(4-Benzylpiperazin-1-ylmethyl)-2,3-dihydro-5H-oxazolo(2,3-b]quinazolin-5-one and -2,³-dihydro-5H-thiazolo(2,3-b]quinazolin-5-one, J. Heterocyclic Chem. 27 (1990) 1467-1472; Kampe, K. -D.: Eine einfache Synthese von 5-Oxo-2,3-dihydro-5H-(1,3]oxazolo[2,3-b]chinazolinen (a simple synthesis-of 5-oxo-2,3-dihydro-5H-[1,3]oxazolo[2,3-b]quianzolines), Synthesis 1976, 469-47).

### EXAMPLE 1

Preparation of (R,S)-2-Mercaptomethyl-2,3-dihydro-5H-thiazolo(2,3-b]-quinazoline-5-one

formula Ia, R1=R2=R3=R4=hydrogen, X=carbonyl, Y=sulfur)

10 mmol of the (R,S)-2-bromomethyl-3H,5H-thiazolo[2,3-b]quinazoline-5-one compound known in the literature, general formula IIa, R1=R2=R3=R4=hydrogen, X=carbonyl, Y=sulfur, Z=bromine, are added to 30 ml monomethylglycol and heated with 12 mmol thiourea under TLC and/or HPLC control at a bath temperature of about 100° C. until the reaction is complete. If while cooling down no crystallization occurs, acetic ester will be added until turbidity appears and the then precipitating isothiuronium salt will be separated. The latter is saponified in 1 N NaOH by the addition of some ethanol and under a nitrogen atmosphere with a bath temperature of 50° C. The course of the reaction is followed by means of TLC or HPLC chromatography. When the reaction is complete, 1 N HCl is used for the acidification while ice cooling is carried out, the precipitate forming is sucked off, washed with water, dried and, when required, recrystallized from ethanol or acetic ester/heptane.

Colorless crystals

F (fixed point): 104-108° C.

Yield: 74%

C₁₁H₁₀N₂OS₂ (250)

IR (υ in cm⁻¹); 1605 (C═N), 1662(C═O)

MS m/e (% B) M⁺250 (51), 203 (100); 162 (20)

### EXAMPLE 2

(R,S)-2-mercaptomethyl-2-methyl-2,3-dihydro-5H-thiazolo[2,3-b]quinazoline-5-one

formula Ia, R1=R3=R4=hydrogen, R2=methyl, X=carbonyl, Y=sulfur)

10 mmol of (R,S)-2-bromomethyl-2-methyl-3H,5H-thiazolo(2,3-b]quinazoline-5-one obtained from the educt known in the literature of Example 1, general formula IIa, R1=R3=R4=hydrogen, R2=methyl, X=carbonyl, Y=sulfur, Z=bromine (colorless crystals; F: 116-118° C. (methanol)), are reacted in analogy to the instructions of Example 1.

Colorless crystals

F: 110-114° C. (ethanol)

Yield: 54%

C₁₂H₁₂N₂OS₂ (264.4)

IR (υ in cm⁻¹): 1608 (C═N), 1672 (C═O) 2568 (SH)

MS m/e (% B): M⁺264(32), 217(100), 162(22)

### EXAMPLE 3

(R,S)-8-carboxy-2-mercaptomethyl-2,3-dihydro-5H-thiazolo [2,3-b]quinazoline-5-one

formula Ia, R1=8-carboxy, R2=R3=R4=hydrogen, X=carbonyl, Y=sulfur)

10 mmol of (R,S)-2-bromomethyl-8-methoxycarbonyl-3H,5H-thiazolo[2,3-b]quinazoline-5-one obtained in analogy to the educt known in the literature of Example 1, general formula IIa, R1=8-methoxycarbonyl, R2=R3=R4=hydrogen, X=carbonyl, Y=sulfur, Z=bromine (ochre cye, F: 204-208° C.), are reacted in analogy to the instructions of Example 1.

Light yellow solid.

F: 214-218° C. (ethanol)

Yield: 42%

C₁₂H₁₀N₂O₃S₂ (294)

IR (υ in cm⁻¹): 1552 (C═N), 1694 (C═O), 2551 (SH)

MS m/e (% B): M⁺294(56), 247 (100), 203 (28)

### EXAMPLE 4

(R,S)-3-mercaptomethyl-2,3-dihydro-5H-thiazolo[2,3-b]quinazoline-5-one

formula Ib, R1=R2=hydrogen, X=carbonyl, Y=sulfur)

10 mmol of (R,S)-3-bromomethyl-3H, 5H-thiazolo (2,3-b]quinazoline-5-one known in the literature, general formula IIb, R1=R2=R3=hydrogen, X=carbonyl, Y=sulfur, Z=bromine, are reacted in analogy to the instructions of Example 1.

Colorless crystals

F: 95-98° C. (ethanol)

Yield: 33%

C₁₁H₁₀N₂CS₂ (250)

IR (υ in cm.⁻¹); 1584 (C═N), 1661 (C═O), 2449 (SH)

MS m/e (% B); M⁺250(50), 217(20), 203(100), 178(100)/*

### EXAMPLE 5

(R,S)-8-chloro-2-mercaptomethyl-2,3-dihydro-5H-thiazolo(2,3-b]quinazoline-5-one

formula Ia, R1=8-chloro, R2=R3=R4=hydrogen, X=carbonyl, Y=sulfur)

10 mmol of (R,S)-2-bromomethyl-8-chloro-3H,5H-thiazolo(2,3-b]quinazoline-5-one obtained in analogy to the educt known in the literature of Example 1, general formula IIa, R1=8-chlorine, R2=R3=R4=hydrogen, X=carbonyl, Y=sulfur, Z=bromine (light yellow solid, F: 204-208° C.), are reacted in analogy to the instructions of Example 1.

Colorless solid.

F: 134-138° C. (ethanol)

Yield: 56%

C₁₁H₉N₂OS₂C (284)

IR (in cm⁻¹) 1574 (C═N), 1673 (C═O)

MS m/e (% B): M⁺284 (60), 251(21), 237(100) 202(29)

The MMP-inhibitory effect of the substances is determined as follows:

Inhibition of the Matrix Metalloproteinase-2 (MMP-2)

MMP-2 (gelatinase) is supplied by cultivated dermal fibroblasts to the culture medium in considerable amounts and is thus easily accessible. The secreted and inactive proform of the enzyme can be converted into the enzymatically active form by trypsin activation or by treatment with organic mercury compounds.

For this purpose, human dermal fibroblasts are obtained according to established standard methods and cultivated and the cell-free culture supernatant is treated with trypsin. Trypsin is then inactivated with a specific inhibitor (TLCK) and active MMP-2 is partially purified by affinity chromatography on gelatin sepharose and subsequent gel filtration on sepharose. MMP-2 was identified and characterized by the availability of a commercial immunoassay.

Preparation of the Human Collagenase MMP-9

Native MMP-9 was obtained reproducibly and with good yield and purity from human Buffy Coat. For this purpose, Buffy Coat is brought to a final concentration of 0,4% using 10% (v/v) triton X-100, shaken on ice for 30 min. and then 1 vol. double binding buffer (40 mM Tris-HCl, pH 7.5, 10 mM CaCl₂, 1 M NaCl, 0.2% (v/v) triton X-100) is added and shaking on ice is continued for another 30 min.

The solution is centrifuged off at 16,000 rpm on an SS-34 rotor at 4° C. for 15 min. and filtered on glass wool. The filtrate is batched with gelatin agarose equilibrated with binding buffer and shaken on ice for 1 hour.

The charged gelatin agarose is transferred to a column and rinsed in protein-free manner using at least 10 vol. binding buffer. The bound MMP-9 is eluted with 2 gel volumes binding buffer plus 5% (v/v) DMSO.

For an exchange of buffers and simultaneous separation of minor MMP-2 contaminations the eluate can be separated on sephadex G-75 by means of gel filtration. Buffer I (20 mM Tris-HCl, pH 7.5, 5 mM CaCl₂, 100mM NaCl, 0.1% (v/v) triton X-100) is used for this purpose.

The resulting eluate contains MMP-9 in the three known configurations: monomer, homodimer, heterodimer. The purity of the enzyme is about 90%, the rest of the foreign proteins being fibronectin and extremely small amounts of TIMPs.

The latent enzyme is activated by incubation at 37° C. using 1/100 vol. trypsin (10 mg/ml), for 30-60 min. Trypsin is inhibited by adding 1 mM PMSF or with a specific inhibitor (TLCK).

Cloning and Expression of the Catalytic MMP-8 Domain

The catalytic MMP-8 domain was used as another test enzyme because it has a high degree of stability and is also available as an active enzyme and therefore needs not be activated, which is in consideration as one of the most frequent causes of error since the mercury compounds used for the activation often interfere with the test system and/or the enzyme and in this way can falsify the measuring results. The cloning strategy was directed towards the circumstance that instead of the whole enzyme only its enzymatically active catalytic domain was cloned into E. coli. By means of the constructed catalytic MMP-8 domain a stable, enzymatically active and highly pure enzyme was obtained which was very well suited for the routine tests of the inhibitory activity of the synthesized inhibitors and the measuring results of the individual series of measurements were absolutely comparable.

The cloning and expression of the recombinant catalytic MMP-8 domain was carried out in accordance with the statements made by SCHNIERER et al. (Schnierer S., Kleine T., Gote T., Hillemann A., Knäuper V., Tschesche H.: The recombinant catalytic domain of human neutrophil collagenase lacks type I collagen substrate specificity. Biochem Biophys Res Comm (1993), vol. 191, No. 2, 319-326).

Quantitative Fluorescence Assay for Matrix Metalloproteinases

The fluorescent group Mca is separated from the internal quencher Dpa by enzymatic cleavage of the synthetic substrate Mca-Pro-Leu-Gly-Leu-Dpa-Ala-Arg-NH₂ using the respective collagenase. This is accompanied by a great increase in fluorescence in the measuring batch, which can be quantified using a fluorimeter (λₑₓ 328 nm, λₑₘ 393 nm) and proceeds linearly within the first few minutes. A certain specificity of the test to matrix metalloproteinases follows from the amino acid sequence-Pro-Leu-Gly-Leu-in the substrate, on the one hand, and from the select incubation conditions, on the other hand. Matrix metalloproteinases cleave the substrate at the Gly-Leu bond. The proteolytic residual activity of pre-incubated batches of enzyme and inhibitor is measured, the substrate and enzyme concentrations having been kept constant and the inhibitor concentration having been varied. Three series of measurements using a different substrate concentration were made for each tested inhibitor. The enzyme activity is calculated in fluorescence units per min. on the basis of the time-sensitive fluorescence increase. The Kᵢ values were determined graphically according to the DIXON method (1953) by applying the reciprocal reaction rate 1/v (y axis) against inhibitor concentration (x axis).

Assay

1984 μl measuring buffer (100 mM Tris-HCl, pH 7.5, 100 mM NaCl, 10 mM CaCl₂, 0.05% brij 35)

2 μl inhibitor dissolved in DMSO, or DMSO alone (non-inhibitory approach)

4 μl enzyme (MMP-2 or MMP-9 or catalytic domain of MMP-8)

5 min. preliminary incubation of the enzyme-inhibitor mixture at room temperature while stirring

start of the reaction with 10 μl substrate dissolved in DMSO

recording of the time-sensitive fluorescence increase over a period of 2 min.

Inhibition of human MMPs by the inhibitors of general formulae Ia and Ib according to the invention

| Compound | | | | |
|---|---|---|---|---|
| according | MMP-2 | MMP-8 | MMP-9 | MT1-MMP |
| to Example | (Kᵢ value) | (Kᵢ value) | (Kᵢ value) | (Kᵢ value) |
| 1 | 70% | 80% | 0.5 pH | 50% inhi- |
| | inhibition | inhibition | | bition with |
| | with 10 μM* | with 10 μM* | | 2.5 μM* |
| 2 | 50% | 85% | 1.0 μM | 4.0 μM |
| | inhibition | inhibition | | |
| | with 10 μM* | with 10 μM* | | |
| 3 | 25% | 24 μM | 15μM | 21 μM |
| | inhibition | | | |
| | with 10 μM* | | | |
| 4 | 50% | 25% | 75% | 55 μM |
| | inhibition | inhibition | inhibition | |
| | with 10 μM* | with 10 μM* | with 10 μM | |
| 5 | 80% | 90% | 85% | 85% |
| | inhibition | inhibition | inhibition | inibition |
| | with 10 μM* | with 10 μM* | with 10 μM* | with 10 μM* |

*) it is not possible to determine an explicit Kᵢ value since the dixon plot is non-linear

1. Thiazolo(2,3-b]quinazolinones, oxazolo[2,3-b]-quinazolinones, imidazo[2,1-b]quinazolinones of general formulae Ia and Ib 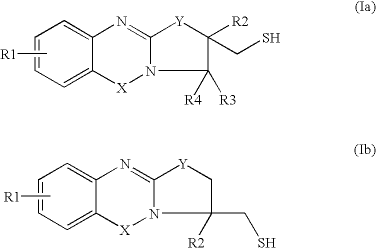
wherein
R1=H, methyl, methoxy, dimethoxy, Hal (Hal=F, Cl, Br), carboxy,
R2, R3, R4=hydrogen, methyl
X=carbonyl, sulfonyl,
Y S, O, NH
and the tautomers and salts thereof.

2. A method of producing the compounds according to claim 1,
characterized in that:
compounds of general formulae IIa and IIb 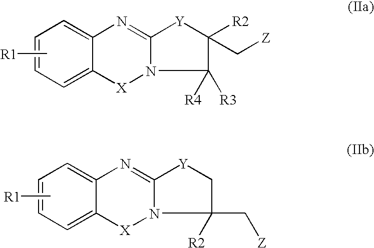
wherein
R1=H, methyl, methoxy, dimethoxy, Hal (Hal=F, Cl, Br), carboxy
R2, R3, R4=hydrogen, methyl
X=carbonyl, sulfonyl
Y=S, O, NH
Z=Cl, Br, J, tosyl
are reacted with a sulfur-transmitting agents in an inert solvent,
the resulting product is isolated
and the desired end product is obtained by subsequent mild saponification

3. The method according to claim 2, characterized in that thiourea or xanthogenates are user as sulfur-transmitting agent.

4. Use or compounds according to claim 1 as matrix metalloproteinase inhibitors.

5. Pharmaceutical composition, containing compounds according to claim 1.
